(19) United States
(12) Patent Application Publication
(10) Pub. No.: US 2014/0005624 A1
(43) Pub. Date: Jan. 2, 2014

(54) Film Containing a Polyalkylene Carbonate

(71) Applicants: James Hongxue Wang, Appleton, WI (US); Gregory James Wideman, Menasha, WI (US)

(72) Inventors: James Hongxue Wang, Appleton, WI (US); Gregory James Wideman, Menasha, WI (US)

(73) Assignee: KIMBERLY-CLARK WORLDWIDE, INC., Neenah, WI (US)

(21) Appl. No.: 13/534,690

(22) Filed: Jun. 27, 2012

(51) Int. Cl. C08L 23/08; B32B 27/32; A61L 15/26; B32B 1/00

(52) U.S. Cl. 604/372; 525/185; 428/35.5; 428/220

(57) ABSTRACT

A film is provided including from about 10 wt. % to about 90 wt. % of at least one polyalkylene carbonate and from about 10 wt. % to about 90 wt. % of at least one polyolefin. The film can be utilized as a packaging film, or as in the construction of an absorbent article (e.g., as the outer cover/backsheet of the article). The film can be a multi-layered film including a core layer that constitutes from about 20% to about 90% of the thickness of the film and an outer layer positioned adjacent to the core layer, with the core layer including from about 10 wt. % to about 90 wt. % of at least one polyalkylene carbonate and from about 10 wt. % to about 90 wt. % of at least one polyolefin. The outer layer can contain about 50 wt. % or more of at least one polyolefin.

### BACKGROUND OF THE INVENTION

Petroleum resources have become more scarce and expensive in recent years, which has further increased the need for environmentally sustainable films which are derived from sustainable resources. Recent advances in catalytic science have led to the polymerization of climate-harmful CO₂ into biodegradable polymers: polyalkylene carbonates (PAC). The most common PAC are polyethylene carbonate (PEC), polypropylene carbonate (PPC), and polybutylene carbonate (PBC). PAC can contain a significant amount of carbon dioxide, for example, PPC contains about 43% by weight of fixed carbon dioxide. Besides environmental benefits, carbon dioxide is also a low cost and abundant carbon source. Converting of carbon dioxide into biodegradable polymers has provided an opportunity to utilize this greenhouse gas to make useful polymer materials. Unfortunately, PAC polymers have a common deficiency of relatively low glass transition temperatures, making them not readily useful for practical applications in pure forms. As such, a need currently exists for a film that contains a PAC polymer, but is nevertheless melt processable and capable of achieving good properties.

### SUMMARY OF THE INVENTION

In one embodiment, a film is generally provided that includes from about 10 wt. % to about 90 wt. % of at least one polyalkylene carbonate and from about 10 wt % to about 90 wt. % of at least one polyolefin. The film can be utilized as a packaging film (e.g., forming a wrap, pouch, or bag), or as in the construction of an absorbent article (e.g., as the outer cover/backsheet of the article).

In one particular embodiment, a multi-layered film having a thickness of about 250 micrometers or less is generally provided. The film can include a core layer that constitutes from about 20% to about 90% of the thickness of the film and an outer layer positioned adjacent to the core layer, with the core layer including from about 10 wt % to about 90 wt % of at least one polyalkylene carbonate and from about 10 wt % to about 90 wt. % of at least one polyolefin. The outer layer can contain about 50 wt. % or more of at least one polyolefin.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a schematic illustration of an exemplary method for forming a multilayer film according to one embodiment of the present invention;

FIG. 2 shows a graph of the peak stress in the machine direction as a function of the film composition;

FIG. 3 shows a DSC thermogram of LLDPE/PPC blend films from the 1st heat cycle according to the Examples;

FIG. 4 shows an SEM image of the cross section of the LLDPE/PPC 80:20 film according to one Example;

FIG. 5 shows an SEM image of the cross section of the LLDPE/PPC 60:40 film according to one Example;

FIG. 6 shows an SEM image of the cross section of the LLDPE/PPC 40:60 film according to one Example; and

FIG. 7 shows an SEM image of the cross section of the LLDPE/PPC 20:80 film according to one Example.

Repeat use of reference characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a film that contains a combination of one or more polyalkylene carbonates that are both biodegradable and renewable and a polyolefin. Despite being biodegradable and renewable, many polyalkylene carbonates tend to be relatively tacky at room temperature, due to their relatively low glass transition temperature in a pure form. For example, polypropylene carbonate are amorphous polymers having a glass transition temperature of about 40° C., while polyethylene carbonate has glass transition temperature of about 25° C. Due to these properties, it was conventionally thought that such polyalkylene carbonates could not be readily formed into useful films since the low glass transition films tend to stick together and become inseparable (blocking). The present inventors have discovered, however, that through selective control over the components, the presently disclosed film composition has overcome the blocking issue during use and storage while remaining to be useful. Among other things, this is accomplished by blending the polyalkylene carbonate(s) with at least one polyolefin. It was also surprisingly found that the inventive films have synergistic properties such as peak stress unexpected from the properties of pure polyalkylene carbonate and polyolefin.

The film can be employed as a single layer film (i.e., free from additional layers), or a multi-layer film. For example, in a multi-layer film, the core layer can include both at least one polyalkylene carbonate and at least one polyolefin, while a polyolefin is also employed in an outer layer attached and/or laminated thereto. In addition to providing functionality to the film (e.g., heat sealing, printing, etc.), the polyolefin-containing outer layer can also help counteract the softness of the polyalkylene carbonates in the core layer, and can help improve processability. In another embodiment, the outer film layers can also include at least one polyalkylene carbonate and at least one polyolefin, the ratio of polyalkylene carbonate to polyolefin in the outside layers can be different from the ratio of polyalkylene carbonate to polyolefin in the core layer. In yet another embodiment, two or more layer of the multi-layer films can have an identical composition. In yet another embodiment, all the layers have the same composition.

In this regard, various embodiments of the present invention will now be described in more detail below.

### I. Film Layer

As indicated above, the film layer contains a blend of at least one polyalkylene carbonate and at least one polyolefin. Typically, the amount of polyalkylene carbonate employed in the film layer is selectively controlled to achieve a balance of biodegradability and ductility. The polyalkylene carbonate may, for example, constitute from about 10 wt % to 90 wt %, in some embodiments from about 50 wt % to 90 wt %, and in some embodiments, from about 60 wt % to about 85 wt. % of the polymer content of the film layer. Likewise, polyolefins typically constitute from about 10 wt. % to about 90 wt. %, in some embodiments from about 10 wt. % to about 50 wt. %, and in some embodiments, from about 15 wt % to about 40 wt. % of the polymer content of the film layer.

Although polyolefins are normally chemically incompatible with polyalkylene carbonates due to their different polarities, the present inventors have discovered that unexpected phase microstructure may be obtained by selectively controlling certain aspects of the film, such as the nature and concentration of the polyolefin. For instance, the films can exhibit unique polymer morphologies based upon the relative amounts of the polyalkylene carbonate and the polyolefin. In one embodiment, where the polyalkylene carbonate is at about 20 wt. % or less of the total weight amount of the PAC and the polyolefin, the PAC forms dispersed domains within a continuous phase of polyolefin. Surprisingly, however, the film exhibits a co-continuous phase structure when the PAC content is from about 40 wt % (67% by volume) to about wt. 60 wt % (48% by volume) of the total weight amount of the PAC and the polyolefin. As used herein, the term “co-continuous” phase structure refers to the topological condition, in a phase-separated, two-component mixture, in which a continuous path through either phase domain may be drawn to all phase domain boundaries without crossing any phase domain boundary.

A. Polyalkylene Carbonates

As stated, the film can include a polyalkylene carbonate (PAC). Generally, PAC is a copolymer of carbon dioxide and at least one alkylene oxide made by reacting the monomers in presence of a suitable catalyst (e.g. a zinc carboxylate catalyst). Particularly suitable alkylene oxides for use as the at least one alkylene oxide monomer include, but are not limited to, ethylene oxide, propylene oxide, cyclopentene oxide, cyclohexene oxide, cis-2-butene oxide, styrene oxide, epichlorohydrin, or mixtures thereof.

As such, the resulting PAC can be a homopolymer or a copolymer of more than one alkylene oxide monomer. Suitable polyalkylene carbonate structures can include repeating alkylene carbonate structure units with 3 to 22 carbonate atoms. Thus, the PAC homopolymers can include, but are not limited to, polyethylene carbonate, polypropylene carbonate, polybutylene carbonate, polyhexylene carbonate, etc. PAC copolymers can include two or more different alkylene carbonate structural units (i.e., monomers), such as polyethylene carbonate-co-propylene carbonate, etc. In yet another embodiment, the PAC can be a copolymer of at least one alkylene oxide monomer with other monomer units (e.g., esters, ethers, amide, etc.).

In certain embodiments, the co-polymerization of the alkylene oxide and carbon dioxide can be achieved via heating the alkylene oxide in a solvent at about 40° C. to about 150° C. (e.g., about 60° C. to about 120° C.) for a suitable time in the presence of carbon dioxide and the catalyst(s). The carbon dioxide can be added to the polymerization reaction in a wide range of pressures. However, the pressure of the carbon dioxide is, in one embodiment, at least 100 psig in order to have a useful rate of polymerization. The upper limit of carbon dioxide pressure is limited only by the equipment in which the polymerization is run.

Several catalyst systems are known that catalyze the copolymerization of carbon dioxide and at least one alkylene oxide, such as zinc carboxylate catalysts (e.g., zinc malonate, zinc succinate, zinc glutarate, zinc adipate, zinc hexafluoroglutarate, zinc pimelate, zinc suberate, zinc azelate, zinc sebacate, or mixtures thereof) as described in U.S. Pat. No. 4,789,727, which is incorporated by reference herein. Additional catalysts and systems are disclosed in U.S. Patent Application Publication No. 2011/0309539 of Steinke, et al., U.S. Pat. No. 6,815,529 of Zhao, et al., U.S. Pat. No. 6,599,577 of Zhao, et al., U.S. Publication No. 2002/0082363 of Zhao, et al., U.S. Publication No. 2011/0152497 of Allen, et al., and U.S. Publication No. 2011/0230580 of Allen, et al., all of which are incorporated by reference herein.

The resulting PAC polymer may contain both ether and carbonate linkages in its main chain. The percentage of carbonate linkages can be dependent on a variety of factors, including the reaction conditions and the nature of the catalyst. In one particular embodiment, for example, the PAC polymer can have more than about 85% of carbonate linkages of all linkages between former alkylene oxide monomers.

In certain embodiments, the PAC in the film can have a number average molecular weight (Mₙ) from about 20,000 to about 200,000 g/mol (e.g., from about 30,000 to 100,000 g/mol, such as from about 35,000 to about 80,000 g/mol).

Additionally, the PAC can have a weight average molecular weight (“M_{w}”) ranging from about 50,000 to about 300,000 grams per mole, in some embodiments from about 70,000 to about 200,000 grams per mole, and in some embodiments, from about 800,000 to about 150,000 grams per mole. The ratio of the weight average molecular weight to the number average molecular weight (“M_{w}/Mₙ”), i.e., the “polydispersity index”, is also relatively low. For example, the polydispersity index typically ranges from about 1.0 to about 4.0, in some embodiments from about 1.2 to about 3.0, and in some embodiments, from about 1.4 to about 2.0. The weight and number average molecular weights may be determined by methods known to those skilled in the art.

The melt flow index (MI) of the polyalkylene carbonate may generally vary, but is typically in the range of about 0.1 grams per 10 minutes to about 100 grams per 10 minutes, in some embodiments from about 0.5 grams per 10 minutes to about 30 grams per 10 minutes, and in some embodiments, about 1 to about 10 grams per 10 minutes, determined at 190° C. The melt flow index is the weight of the polymer (in grams) that may be forced through an extrusion rheometer orifice (0.0825-inch diameter) when subjected to a force of 2160 grams in 10 minutes at 190° C., and may be determined in accordance with ASTM Test Method D1238-E.

One particularly suitable polyalkylene carbonates for inclusion in the film is polypropylene carbonate (PPC) available from Inner Mongolia Mengxi High-Tech Group Co., Ltd., under the brand name Melicsea polypropylene carbonate (e.g., MXJJ-001 with a melt flow of 3.6 g/10 minutes at 150° C.).

B. Polyolefins

As indicated above, a polyolefin is also employed in the film. Among other things, the polyolefin helps to counteract the low glass transition temperature of the polyalkylene carbonates, thereby improving mechanical properties and melt processability of the film. Exemplary polyolefins for this purpose may include, for instance, polyethylene, polypropylene, blends and copolymers thereof. In one particular embodiment, a polyethylene is employed that is a copolymer of ethylene and an α-olefin, such as a C₃-C₂₀ α-olefin or C₃-C₁₂ α-olefin. Suitable α-olefins may be linear or branched (e.g., one or more C₁-C₃ alkyl branches, or an aryl group). Specific examples include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin co-monomers are 1-butene, 1-hexene and 1-octene. The ethylene content of such copolymers may be from about 60 mole % to about 99 mole %, in some embodiments from about 80 mole % to about 98.5 mole %, and in some embodiments, from about 87 mole % to about 97.5 mole %. The α-olefin content may likewise range from about 1 mole % to about 40 mole %, in some embodiments from about 1.5 mole % to about 15 mole %, and in some embodiments, from about 2.5 mole % to about 13 mole %.

The density of the polyethylene may vary depending on the type of polymer employed, but generally ranges from 0.85 to 0.96 grams per cubic centimeter (“g/cm³”). Polyethylene “plastomers”, for instance, may have a density in the range of from 0.85 to 0.91 g/cm³. Likewise, “linear low density polyethylene” (“LLDPE”) may have a density in the range of from 0.91 to 0.940 g/cm³; “low density polyethylene” (“LDPE”) may have a density in the range of from 0.910 to 0.940 g/cm³; and “high density polyethylene” (“HDPE”) may have density in the range of from 0.940 to 0.970 g/cm³. Densities may be measured in accordance with ASTM 1505. Particularly suitable ethylene-based polymers for use in the present invention may be available under the designation EXACT™ from ExxonMobil Chemical Company of Houston, Tex. Other suitable polyethylene plastomers are available under the designation ENGAGE™ and AFFINITY™ from Dow Chemical Company of Midland, Mich. Still other suitable ethylene polymers are available from The Dow Chemical Company under the designations DOWLEX™ (LLDPE) and ATTANE™ (ULDPE). Other suitable ethylene polymers are described in U.S. Pat. Nos. 4,937,299 to Ewen et al.; 5,218,071 to Tsutsui et al.; 5,272,236 to Lai, et al.; and 5,278,272 to Lai, et al., which are incorporated herein in their entirety by reference thereto for all purposes.

Of course, the present invention is by no means limited to the use of ethylene polymers. For instance, propylene polymers may also be suitable for use as a semi-crystalline polyolefin. Suitable propylene polymers may include, for instance, polypropylene homopolymers, as well as copolymers or terpolymers of propylene with an α-olefin (e.g., C₃-C₂₀), such as ethylene, 1-butene, 2-butene, the various pentene isomers, 1-hexene, 1-octene, 1-nonene, 1-decene, 1-unidecene, 1-dodecene, 4-methyl-1-pentene, 4-methyl-1-hexene, 5-methyl-1-hexene, vinylcyclohexene, styrene, etc. The comonomer content of the propylene polymer may be about 35 wt. % or less, in some embodiments from about 1 wt. % to about 20 wt. %, and in some embodiments, from about 2 wt. % to about 10 wt %. The density of the polypropylene (e.g., propylene/α-olefin copolymer) may be 0.95 grams per cubic centimeter (g/cm³) or less, in some embodiments, from 0.85 to 0.92 g/cm³, and in some embodiments, from 0.85 g/cm³ to 0.91 g/cm³. Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes.

Any of a variety of known techniques may generally be employed to form the polyolefins. For instance, olefin polymers may be formed using a free radical or a coordination catalyst (e.g., Ziegler-Natta or metallocene). Metallocene-catalyzed polyolefins are described, for instance, in U.S. Pat. Nos. 5,571,619 to McAlpin et al.; 5,322,728 to Davis et al.; 5,472,775 to Obijeski et al.; 5,272,236 to Lai et al.; and 6,090,325 to Wheat, et al., which are incorporated herein in their entirety by reference thereto for all purposes.

The melt flow index (MI) of the polyolefins may generally vary, but is typically in the range of about 0.1 grams per 10 minutes to about 100 grams per 10 minutes, in some embodiments from about 0.5 grams per 10 minutes to about 30 grams per 10 minutes, and in some embodiments, about 1 to about 10 grams per 10 minutes, determined at 190° C. The melt flow index is the weight of the polymer (in grams) that may be forced through an extrusion rheometer orifice (0.0825-inch diameter) when subjected to a force of 2160 grams in 10 minutes at 190° C., and may be determined in accordance with ASTM Test Method D1238-E.

C. Other Components

One beneficial aspect of the present invention is that a film can be readily formed without the need for compatibilizers or plasticizers conventionally thought to be required to melt process a polyalkylene carbonate. Thus, in certain embodiments, the film layer may be free of such ingredients, which further enhances the overall biodegradability and renewability of the film. Additionally, in one embodiment, the film can be free from other polymeric material. For example, the film can be free from polyesters (including biodegradable polyesters such as polylactic acids, polycaprolactone, polyhydroxyalkanoate, etc.), polyurethanes, etc.

Nevertheless, in some cases, compatibilizer and/or plasticizers may still be employed in the film layer, typically in an amount of no more than about 40 wt. %, in some embodiments from about 0.1 wt. % to about 30 wt %, in some embodiments from about 0.5 wt. % to about 25 wt %, and in some embodiments, from about 1 wt. % to about 15 wt % of the film layer.

When employed, the compatibilizer may be a functionalized polyolefin that possesses a polar component provided by one or more functional groups that is compatible with the polyalkylene carbonates and a non-polar component provided by an olefin that is compatible with the polyolefin. The polar component may, for example, be provided by one or more functional groups and the non-polar component may be provided by an olefin. The olefin component of the compatibilizer may generally be formed from any linear or branched α-olefin monomer, oligomer, or polymer (including copolymers) derived from an olefin monomer. The α-olefin monomer typically has from 2 to 14 carbon atoms and preferably from 2 to 6 carbon atoms. Examples of suitable monomers include, but not limited to, ethylene, propylene, butene, pentene, hexene, 2-methyl-1-propene, 3-methyl-1-pentene, 4-methyl-1-pentene, and 5-methyl-1-hexene. Examples of polyolefins include both homopolymers and copolymers, i.e., polyethylene, ethylene copolymers such as EPDM, polypropylene, propylene copolymers, and polymethylpentene polymers. An olefin copolymer can include a minor amount of non-olefinic monomers, such as styrene, vinyl acetate, diene, or acrylic and non-acrylic monomer. Functional groups may be incorporated into the polymer backbone using a variety of known techniques. For example, a monomer containing the functional group may be grafted onto a polyolefin backbone to form a graft copolymer. Such grafting techniques are well known in the art and described, for instance, in U.S. Pat. No. 5,179,164. In other embodiments, the monomer containing the functional groups may be copolymerized with an olefin monomer to form a block or random copolymer. Regardless of the manner in which it is incorporated, the functional group of the compatibilizer may be any group that provides a polar segment to the molecule, such as a carboxyl group, acid anhydride group, acid amide group, imide group, carboxylate group, epoxy group, amino group, isocyanate group, group having oxazoline ring, hydroxyl group, and so forth. Maleic anhydride and epoxy modified polyolefins are particularly suitable for use in the present invention. Such modified polyolefins are typically formed by grafting maleic anhydride onto a polymeric backbone material. Such maleated polyolefins are available from E. I. du Pont de Nemours and Company under the designation Fusabond®, such as the P Series (chemically modified polypropylene), E Series (chemically modified polyethylene), C Series (chemically modified ethylene vinyl acetate), A Series (chemically modified ethylene acrylate copolymers or terpolymers), or N Series (chemically modified ethylene-propylene, ethylene-propylene diene monomer (“EPDM”) or ethylene-octene). Alternatively, maleated polyolefins are also available from Chemtura Corp. under the designation Polybond® and Eastman Chemical Company under the designation Eastman G series, and AMPLIFY™ GR Functional Polymers (maleic anhydride grafted polyolefins). Epoxy-containing compatibilizers include olefin-acrylate-glycidyl(meth)acrylate terpolymers such as ethylene-methyl ethyl acrylate terpolymer, ethylene-methyl acrylate-glycidyl methacrylate such as Lotador® AX 8840, AX 8900 (melt flow index: 6 g/10 min, methyl acrylate content 24%, glycidyl methacrylate content 8%), AX 8950 (melt flow index: 81 g/10 min, methyl acrylate content 24%, glycidyl methacrylate content 8%), CX 8902, CX 8904, etc.

Likewise, when employed, suitable plasticizers may include polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, and sorbitol), polyols (e.g., ethylene glycol, glycerol, propylene glycol, dipropylene glycol, butylene glycol, and hexane triol), etc. Also suitable are hydrogen bond-forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as copolymers of ethylene and acrylic acid, polyethylene grafted with maleic acid, polybutadiene-co-acrylic acid, polybutadiene-co-maleic acid, polypropylene-co-acrylic acid, polypropylene-co-maleic acid, and other hydrocarbon based acids. A low molecular weight plasticizer is preferred, such as less than about 20,000 g/mol, preferably less than about 5,000 g/mol and more preferably less than about 1,000 g/mol.

Besides the components noted above, still other additives may also be incorporated into the film, such as melt stabilizers, dispersion aids (e.g., surfactants), processing aids (PPA) or stabilizers, heat stabilizers, light stabilizers, antioxidants, heat aging stabilizers, whitening agents, antiblocking agents, bonding agents, lubricants, fillers, anti-static additives, etc.

### II. Optional Outer Layer

Additionally, the present inventors have discovered that the typical islands-in-the-sea morphology that would be normally expected from a polymer blend of a polar polyalkylene carbonate(s) and non-polar polyolefin can be replaced by novel co-continuous morphology which exhibited new mechanical properties. Employing an outer layer can also further enhance the physical and mechanical properties of the film.

As indicated above, the outer layer of the multi-layered film can contain, in one embodiment, at least one polyolefin. In addition to providing functionality to the film (e.g., heat sealing, printing, etc.), the outer layer also helps counteract the softness of the polyalkylene carbonate in the core layer, and helps improve processability. Exemplary polyolefins for this purpose may include, for instance, polyethylene, polypropylene, blends and copolymers thereof, such as described above. Ethylene copolymers are particularly suitable for use in the outer layer, such as LDPE, LLDPE, polyethylene plastomers, single-site catalyzed polyolefins (e.g., metallocene-catalyzed), ethylene vinyl acetate copolymers, ethylene acrylic acid copolymers, ethylene methacrylic acid copolymers, ethylene methyl acrylate copolymers, ethylene butyl acrylate copolymers, ethylene vinyl alcohol copolymers, etc.

To help ensure that the desired properties are achieved, polyolefins constitute at least the majority of the outer layer, such as about 50 wt. % or more, in some embodiments about 60 wt. % or more, and in some embodiments, about 75 wt. % or more. In certain embodiments, for example, polyolefins may constitute the entire polymer content of the outer layer. In other embodiments, however, it may be desired to incorporate one or more additional polymers in the outer layer that are biodegradable, renewable, or both, typically in an amount of no more than about 50 wt. %, in some embodiments from about 1 wt. % to about 45 wt. %, and in some embodiments, from about 5 wt. % to about 40 wt % of the polymer content of the outer layer.

When employed in the outer layer, the additional polymers may include any of the polymers referenced above. In addition to those noted above, another suitable polymer that may be employed in the outer layer is a starch layer, which can be both biodegradable and renewable. Although starch polymers are produced in many plants, typical sources includes seeds of cereal grains, such as corn, waxy corn, wheat, sorghum, rice, and waxy rice; tubers, such as potatoes; roots, such as tapioca (i.e., cassava and manioc), sweet potato, and arrowroot and the pith of the sago palm. Broadly speaking, any native (unmodified) and/or modified starch (e.g., chemically or enzymatically modified) may be employed in the present invention. Chemically modified starches may, for instance, be obtained through typical processes known in the art (e.g., esterification, etherification, oxidation, acid hydrolysis, enzymatic hydrolysis, etc.). Starch ethers and/or esters may be particularly desirable, such as hydroxyalkyl starches, carboxymethyl starches, etc. The hydroxyalkyl group of hydroxylalkyl starches may contain, for instance, 2 to 10 carbon atoms, in some embodiments from 2 to 6 carbon atoms, and in some embodiments, from 2 to 4 carbon atoms. Representative hydroxyalkyl starches such as hydroxyethyl starch, hydroxypropyl starch, hydroxybutyl starch, and derivatives thereof. Starch esters, for instance, may be prepared using a wide variety of anhydrides (e.g., acetic, propionic, butyric, and so forth), organic adds, acid chlorides, or other esterification reagents. The degree of esterification may vary as desired, such as from 1 to 3 ester groups per glucosidic unit of the starch.

The starch polymer may contain different weight percentages of amylose and amylopectin, different polymer molecular weights, etc. High amylose starches contain greater than about 50% by weight amylose and low amylose starches contain less than about 50% by weight amylose. Although not required, low amylose starches having an amylose content of from about 10% to about 40% by weight, and in some embodiments, from about 15% to about 35% by weight, are particularly suitable for use in the present invention. Examples of such low amylose starches include corn starch and potato starch, both of which have an amylose content of approximately 20% by weight. Particularly suitable low amylose starches are those having a number average molecular weight (“Mₙ”) ranging from about 50,000 to about 1,000,000 grams per mole, in some embodiments from about 75,000 to about 800,000 grams per mole, and in some embodiments, from about 100,000 to about 600,000 grams per mole, and/or a weight average molecular weight (“M_{w}”) ranging from about 5,000,000 to about 25,000,000 grams per mole, in some embodiments from about 5,500,000 to about 15,000,000 grams per mole, and in some embodiments, from about 6,000,000 to about 12,000,000 grams per mole. The ratio of the weight average molecular weight to the number average molecular weight (“M_{w}/Mₙ”), i.e., the “polydispersity index”, is also relatively high. For example, the polydispersity index may range from about 10 to about 100, and in some embodiments, from about 20 to about 80. The weight and number average molecular weights may be determined by methods known to those skilled in the art.

If desired, a plasticizer may also be employed in the outer layer to further enhance the ability of an additional polymer (e.g., starch polymer, cellulose polymer, etc.) contained therein to be melt processed. For example, such plasticizers can soften and penetrate into the outer membrane of a starch polymer and cause the inner starch chains to absorb water and swell. This swelling will, at some point, cause the outer shell to rupture and result in an irreversible destructurization of starch granules. Once destructurized, the starch polymer chains, which are initially compressed within the granules, may stretch out and form a generally disordered intermingling of polymer chains. Upon resolidification, however, the chains may reorient themselves to form crystalline or amorphous solids having varying strengths depending on the orientation of the starch polymer chains.

A plasticizer may be incorporated into the outer layer using any of a variety of known techniques. For example, polymers may be “pre-plasticized” prior to incorporation into the film to form what is often referred to as a “thermoplastic masterbatch.” The relative amount of the polymer and plasticizer employed in the thermoplastic masterbatch may vary depending on a variety of factors, such as the desired molecular weight, the type of polymer, the affinity of the plasticizer for the polymer, etc. Typically, however, polymers constitute from about 40 wt. % to about 98 wt. %, in some embodiments from about 50 wt % to about 95 wt. %, and in some embodiments, from about 60 wt % to about 90 wt. % of the thermoplastic masterbatch. Likewise, plasticizers typically constitute from about 2 wt. % to about 60 wt. %, in some embodiments from about 5 wt % to about 50 wt. %, and in some embodiments, from about 10 wt % to about 40 wt % of the thermoplastic masterbatch. Batch and/or continuous melt blending techniques may be employed to blend a polymer and plasticizer and form a masterbatch. For example, a mixer/kneader, Banbury mixer, Farrel continuous mixer, single-screw extruder, twin-screw extruder, roll mill, etc., may be utilized. One particularly suitable melt-blending device is a co-rotating, twin-screw extruder (e.g., USALAB twin-screw extruder available from Thermo Electron Corporation of Stone, England or an extruder available from Coperion Werner Pfleiderer from Ramsey, N.J.). Such extruders may include feeding and venting ports and provide high intensity distributive and dispersive mixing. For example, a polymer may be initially fed to a feeding port of the twin-screw extruder. Thereafter, a plasticizer may be injected into the polymer composition. Alternatively, the polymer may be simultaneously fed to the feed throat of the extruder or separately at a different point along its length. Melt blending may occur at any of a variety of temperatures, such as from about 30° C. to about 200° C., in some embodiments, from about 40° C. to about 160° C., and in some embodiments, from about 50° C. to about 150° C.

Alternatively, the other polymers in the outer layer can also contain polylactic acid, polybutylene succinate, polyhydroxyalkanoate, thermoplastic cellulose, etc.

In addition to those mentioned above, other additives may also be employed in the outer layer as is known in the art, such as melt stabilizers, dispersion aids (e.g., surfactants), processing aids or stabilizers, heat stabilizers, light stabilizers, antioxidants, heat aging stabilizers, whitening agents, antiblocking agents, bonding agents, lubricants, fillers, anti-static additives, etc.

### III. Multi-Layer Film Construction

As stated, in the embodiment having multi-layers, the film can contain a core layer (described as the film layer above in section I above) that is positioned adjacent to an outer layer. In addition to these layers, it should be understood that various other layers may also be employed in the multi-layer film. For example, the multi-layer film may contain from two (2) to fifteen (15) layers, and in some embodiments, from three (3) to twelve (12) layers. In one embodiment, for example, the multi-layer film is a two-layered film that contains only the core layer and the outer layer. In another embodiment, the multi-layer film contains more than two layers (e.g., three (3) layers) in which the core layer is positioned between first and second outer layers. In such embodiments, the first outer layer may serve as a heat-sealing layer of the multi-layer film, and the second outer layer may serve as a printable layer. The first outer layer, second outer layer, or both may be formed in the manner described above. For example, polyolefins may constitute at least the majority of the first outer layer and/or second outer layer, such as about 50 wt. % or more, in some embodiments about 60 wt. % or more, and in some embodiments, about 75 wt. % or more. In certain embodiments, for example, polyolefins may constitute the entire polymer content of the first outer layer and/or the second outer layer. In other embodiments, as noted above, one or more additional polymers may be employed in the first outer layer and/or second outer layer that are biodegradable, renewable, or both, typically in an amount of no more than about 50 wt %, in some embodiments from about 1 wt. % to about 45 wt %, and in some embodiments, from about 5 wt. % to about 40 wt. % of the polymer content of the respective outer layer. It should be noted the first and second outer layers may be formed from the same composition (e.g., same type of polyolefins and same concentration of polyolefins, etc.) or from a different composition (e.g., different types of polyolefins and/or different concentration of polyolefins).

Regardless of the number of layers employed, the core layer typically constitutes a substantial portion of the thickness of the multi-layer film, such as from about 20% to about 90%, in some embodiments from about 30% to about 80%, and in some embodiments, from about 40% to about 70% of the thickness of the multi-layer film. On the other hand, the combined thickness of the outer layer(s) is typically from about 10% to about 65%, in some embodiments from about 20% to about 60%, and in some embodiments, from about 25% to about 55% of the thickness of the multi-layer film. When two outer layers are employed, for example, each individual outer layer may constitute from about 5% to about 35%, in some embodiments from about 10% to about 30%, and in some embodiments, from about 12% to about 28% of the thickness of the multi-layer film. The total thickness of the multi-layer film may generally vary depending upon the desired use. Nevertheless, the multi-layer film thickness is typically minimized to increase flexibility and reduce the time needed for the film to degrade. Thus, in most embodiments, the multi-layer film has a total thickness of about 250 micrometers or less, in some embodiments from about 1 to about 200 micrometers, in some embodiments from about 2 to about 150 micrometers, and in some embodiments, from about 5 to about 120 micrometers. For example, when two outer layers are employed, each individual layer may have a thickness of from about 0.5 to about 50 micrometers, in some embodiments from about 1 to about 35 micrometers, and in some embodiments, from about 5 to about 25 micrometers. Likewise, the core layer may have a thickness of from about from about 10 to about 100 micrometers, in some embodiments from about 15 to about 80 micrometers, and in some embodiments, from about 20 to about 60 micrometers.

Despite having such a small thickness, the multi-layer film of the present invention is nevertheless able to retain good mechanical properties during use.

One parameter that is indicative of the relative dry strength of the film is the ultimate tensile strength, which is equal to the peak stress obtained in a stress-strain curve, such as obtained in accordance with ASTM Standard D-5034. Desirably, the film of the present invention exhibits a peak stress (when dry) in the machine direction (“MD”) of from about 10 to about 100 Megapascals (MPa), in some embodiments from about 15 to about 70 MPa, and in some embodiments, from about 20 to about 60 MPa, and a peak stress in the cross-machine direction (“CD”) of from about 2 to about 40 Megapascals (MPa), in some embodiments from about 4 to about 40 MPa, and in some embodiments, from about 5 to about 30 MPa. Additionally, the film can, in one embodiment, have a strain-at-break of from about 400% to about 600% in its machine direction. Likewise, the film can, in one embodiment, have an energy-at-break ranging from 70 to 120 J/cm³ in machine direction.

Although possessing good strength, the film is relatively ductile. One parameter that is indicative of the ductility of the film is the percent strain of the film at its break point, as determined by the stress-strain curve, such as obtained in accordance with ASTM Standard D-5034. For example, the percent strain at break of the film in the machine direction may be about 200% or more, in some embodiments about 250% or more, and in some embodiments, from about 300% to about 800%. Likewise, the percent strain at break of the film in the cross-machine direction may be about 300% or more, in some embodiments about 400% or more, and in some embodiments, from about 500% to about 1000%. Another parameter that is indicative of stiffness is the modulus of elasticity of the film, which is equal to the ratio of the tensile stress to the tensile strain and is determined from the slope of a stress-strain curve. For example, the film typically exhibits a modulus of elasticity (when dry) in the machine direction (“MD”) of from about 50 to about 1200 Megapascals (“MPa”), in some embodiments from about 60 to about 800 MPa, and in some embodiments, from about 100 to about 400 MPa, and a modulus in the cross-machine direction (“CD”) of from about 50 to about 600 Megapascals (“MPa”), in some embodiments from about 60 to about 500 MPa, and in some embodiments, from about 100 to about 400 MPa.

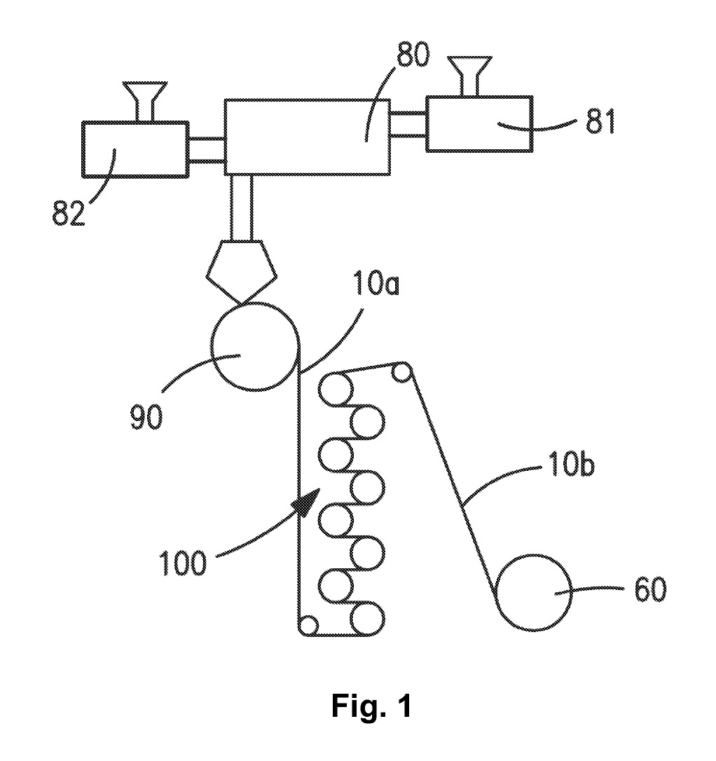

The multi-layered film may be prepared by co-extrusion of the layers, extrusion coating, or by any conventional layering process. Two particularly advantageous processes are cast film coextrusion and blown film coextrusion. In such processes, two or more of the film layers are formed simultaneously and exit the extruder in a multilayer form. Some examples of such processes are described in U.S. Pat. Nos. 6,075,179 to McCormack. et al. and 6,309,736 to McCormack, et al., which are incorporated herein in their entirety by reference thereto for all purposes. Referring to FIG. 1, for instance, one embodiment of a method for forming a co-extruded cast multi-layer film is shown. In the particular embodiment of FIG. 1, the raw materials for the outer layer (not shown) are supplied to a first extruder 81 and the raw material for the core layer (not shown) are supplied to a second extruder 82. The extruders feed the compounded materials to a die 80 that casts the layers onto a casting roll 90 to form a two-layered precursor film 10a. Additional extruders (not shown) may optionally be employed to form other layers of the film as is known in the art. The casting roll 90 may optionally be provided with embossing elements to impart a pattern to the film. Typically, the casting roll 90 is kept at temperature sufficient to solidify and quench the sheet 10a as it is formed, such as from about 20 to 60° C. If desired, a vacuum box may be positioned adjacent to the casting roll 90 to help keep the precursor film 10a close to the surface of the roll 90. Additionally, air knives or electrostatic pinners may help force the precursor film 10a against the surface of the casting roll 90 as it moves around a spinning roll. An air knife is a device known in the art that focuses a stream of air at a very high flow rate to pin the edges of the film.

In addition to casting, other methods may also be used to form the film, such as blowing, flat die extruding, etc. For example, the film may be formed by a blown process in which a gas (e.g., air) is used to expand a bubble of the extruded polymer blend through an annular die. The bubble is then collapsed and collected in flat film form. Processes for producing blown films are described, for instance, in U.S. Pat. No. 3,354,506 to Raley: U.S. Pat. No. 3,650,649 to Schippers; and U.S. Pat. No. 3,801,429 to Schrenk et al., as well as U.S. Patent Application Publication Nos. 2005/0245162 to McCormack. et al. and 2003/0068951 to Boggs, et al.

Regardless of how it is formed, the film may then be optionally oriented in one or more directions to further improve film uniformity and reduce thickness. For example, the film may be immediately reheated to a temperature below the melting point of one or more polymers in the film, but high enough to enable the composition to be drawn or stretched. In the case of sequential orientation, the “softened” film is drawn by rolls rotating at different speeds or rates of rotation such that the sheet is stretched to the desired draw ratio in the longitudinal direction (machine direction). This “uniaxially” oriented film may then be laminated to a fibrous web. In addition, the uniaxially oriented film may also be oriented in the cross-machine direction to form a “biaxially oriented” film. For example, the film may be clamped at its lateral edges by chain dips and conveyed into a tenter oven. In the tenter oven, the film may be reheated and drawn in the cross-machine direction to the desired draw ratio by chain clips, which are diverged in their forward travel.

Referring again to FIG. 1, for instance, one method of forming a uniaxially oriented film is shown. As illustrated, the precursor film 10a is directed to a film-orientation unit 100 or machine direction orienter (“MDO”), such as commercially available from Marshall and Willams, Co. of Providence, R.I. The MDO has a plurality of stretching rolls (such as from 5 to 8) which progressively stretch and thin the film in the machine direction, which is the direction of travel of the film through the process as shown in FIG. 1. While the MDO 100 is illustrated with eight rolls, it should be understood that the number of rolls may be higher or lower, depending on the level of stretch that is desired and the degrees of stretching between each roll. The film may be stretched in either single or multiple discrete stretching operations. It should be noted that some of the rolls in an MDO apparatus may not be operating at progressively higher speeds. If desired, some of the rolls of the MDO 100 may act as preheat rolls. If present, these first few rolls heat the film 10a above room temperature (e.g., to 125° F.). The progressively faster speeds of adjacent rolls in the MDO act to stretch the film 10a. The rate at which the stretch rolls rotate determines the amount of stretch in the film and final film weight. The resulting film 10b may then be wound and stored on a take-up roll 60. While not shown here, various additional potential processing and/or finishing steps known in the art, such as slitting, treating, aperturing, printing graphics, or lamination of the film with other layers (e.g., nonwoven web materials), may be performed without departing from the spirit and scope of the invention.

### IV. Applications

The film of the present invention is particularly suitable for use as a packaging film, such as an individual wrap, packaging pouches, bundle films, or bags for the use of a variety of articles, such as food products, paper products (e.g., tissue, wipes, paper towels, etc.), absorbent articles, etc. Various suitable pouch, wrap, or bag configurations for absorbent articles are disclosed, for instance, in U.S. Pat. Nos. 6,716,203 to Sorebo, et al. and 6,380,445 to Moder, et al., as well as U.S. Patent Application Publication No. 2003/0116462 to Sorebo, et al., all of which are incorporated herein in their entirety by reference thereto for al purposes.

The film may also be employed in other applications. For example, the film may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins, pantiliners, etc.), swim wear, baby wipes, and so forth; medical absorbent articles, such as garments, fenestration materials, underpads, bedpads, bandages, absorbent drapes, and medical wipes; food service wipers; clothing articles; and so forth. Several examples of such absorbent articles are described in U.S. Pat. Nos. 5,649,916 to DiPalma, et al.; 6,110,158 to Kielpikowski; 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. Nos. 4,886,512 to Damico et al.; 5,558,659 to Sherrod et al.; 6,888,044 to Fell et al.; and 6,511,465 to Freiburger et al., all of which are incorporated herein in their entirety by reference thereto for all purposes. The film can be used as a baffle film for feminine care pad and pantiliner, adult incontinent pad baffle film, the outer cover film for infant diaper, child training pants, and adult incontinent pants. Materials and processes suitable for forming such absorbent articles are well known to those skilled in the art.

The present invention may be better understood with reference to the following examples.

### Materials

Polypropylene carbonate (PPC, a CO₂ polymer) from Inner Mongolia Mengxi Hi-Tech Co., Ltd., Wuhai, Inner Mongolia, China. The grade used was Melicsea MXJJ-001 with a melt flow of 3.6 g/10 minutes at 150° C., as an example of polyalkylene carbonate.

Dowlex 2244G (Dow Chemical) is a linear low density polyethylene with a melt flow of 1.0 g/10 minutes at 190° C.

### Comparative Example 1

Dowlex 2244G LLDPE was extruded on a HAAKE single screw extruder with an L/D ratio of 25/1 fitted with a HAAKE 6″ cast film die. The temperatures of the cast film extruder were set at 160, 160, 165, and 170° C. respectively for the three heating zones and die. The screw speed was 50 rpm. The melt pressure was 28 bar, the torque was 18 N·m, the melt temperature was 185° C. The resulting film was soft to the touch and transparent.

### Comparative Example 2

Melicsea PPC was extruded on a HAAKE single screw extruder with an L/D ratio of 25/1 fitted with a HAAKE 6″ cast film die. The temperatures of the cast film extruder were set at 150, 155, 155, and 160° C. respectively for the three heating zones and die. The screw speed was 50 rpm. The melt pressure was 6 bar, the torque was 6 N·m, the melt temperature was 173° C. The resulting film was soft to the touch and transparent. After short aging, the film would block or adhere to itself.

### Example 1

Dowlex 2244G LLDPE and Melicsea PPC were dry blended at 80:20 w/w. The polymer blend was extruded on a HAAKE single screw extruder with an L/D ratio of 25/1 fitted with a HAAKE 6″ cast film die. The temperatures of the cast film extruder were set at 160, 160, 165, and 170° C. respectively for the three heating zones and die. The screw speed was 50 rpm. The melt pressure was 16 bar, the torque was 10 N·m, the melt temperature was 184° C. The resulting film was smooth at surface, it was soft to the touch and transparent. No film blocking was observed.

### Example 2

Dowlex 2244G LLDPE and Melicsea PPC were dry blended at 60:40 w/w. The polymer blend was extruded on a HAAKE single screw extruder with an L/D ratio of 25/1 fitted with a HAAKE 6″ cast film die. The temperatures of the cast film extruder were set at 160, 160, 165, and 170° C. respectively for the three heating zones and die. The screw speed was 50 rpm. The melt pressure was 13 bar, the torque was 9 N·m, the melt temperature was 184° C. The resulting film was smooth at surface, it was soft to the touch and transparent. After short aging, the film would slightly block or adhere to itself.

### Example 3

Dowlex 2244G LLDPE and Melicsea PPC were dry blended at 40:60 w/w. The polymer blend was extruded on a HAAKE single screw extruder with an L/D ratio of 25/1 fitted with a HAAKE 6″ cast film die. The temperatures of the cast film extruder were set at 160, 160, 165, and 170° C. respectively for the three heating zones and die. The screw speed was 50 rpm. The melt pressure was 10 bar, the torque was 9 N-m, the melt temperature was 184° C. The resulting film was smooth at surface, it was soft to the touch and transparent. After short aging, the film would block or adhere to itself.

### Example 4

Dowlex 2244G LLDPE and Melicsea PPC were dry blended at 20:80 w/w. The polymer blend was extruded on a HAAKE single screw extruder with an L/D ratio of 25/1 fitted with a HAAKE 6″ cast film die. The temperatures of the cast film extruder were set at 160, 160, 165, and 170° C. respectively for the three heating zones and die. The screw speed was 50 rpm. The melt pressure was 6 bar, the torque was 7 N·m, the melt temperature was 184° C. The resulting film was smooth at surface, it was soft to the touch and transparent. After short aging, the film would block or adhere to itself.

TABLE 1
Cast Film Process Conditions

| | Speed | Zone 1 Temp. | Zone 2 Temp. | Zone 3 Temp. | Die Temp. | Melt Temp. | Die Pressure | Torque |
|---|---|---|---|---|---|---|---|---|
| | (rpm) | (° C.) | (° C.) | (° C.) | (° C.) | (° C.) | (bar) | (N · m) |
| Comparative | 50 | 160 | 160 | 165 | 170 | 185 | 28 | 18 |
| Example 1 | | | | | | | | |
| Example 1 | 50 | 160 | 160 | 165 | 170 | 184 | 16 | 10 |
| 2244G/Melicsea | | | | | | | | |
| PPC 80:20 | | | | | | | | |
| Example 2 | 50 | 160 | 160 | 165 | 170 | 184 | 13 | 9 |
| 2244G/Melicsea | | | | | | | | |
| PPC 60:40 | | | | | | | | |
| Example 3 | 50 | 160 | 160 | 165 | 170 | 184 | 10 | 9 |
| 2244G/Melicsea | | | | | | | | |
| PPC 40:60 | | | | | | | | |
| Example 4 | 50 | 160 | 160 | 165 | 170 | 184 | 6 | 7 |
| 2244G/Melicsea | | | | | | | | |
| PPC 20:80 | | | | | | | | |

TABLE 2
Cast Film Conditions

| | Speed | Zone 1 Temp. | Zone 2 Temp. | Zone 3 Temp. | Die Temp. | Melt Temp. | Die Pressure | Torque |
|---|---|---|---|---|---|---|---|---|
| | (rpm) | (° C.) | (° C.) | (° C.) | (° C.) | (° C.) | (bar) | (N · m) |
| Comparative | 50 | 150 | 155 | 155 | 160 | 173 | 6 | 6 |
| Example 2 | | | | | | | | |
| PPC | | | | | | | | |

### Example 5

Films were tested for tensile properties using ASTM D638-08 Standard Test Method for Tensile Properties of Plastics. Tensile testing was performed on a Sintech 1/D. Five samples were tested for each film in both the machine direction (MD) and the cross direction (CD). A computer program Test Works 4 was used to collect data during testing and to generate a stress versus strain curve from which a number of properties were determined, including modulus, peak stress, elongation, and toughness.

After conditioning for 24 hours at 70° F.@50% humidity the film samples were cut into dog bone shapes with a center width of 3.0 mm before testing. The dog-bone film samples were held in place using grips on the Sintech device with a gauge length of 18.0 mm. The film samples were stretched at a crosshead speed of 5.0 in/min until breakage occurred.

The tensile testing results are summarized in Table 3 respectively for both MD and CD. Pure PPC had a low peak stress of only 20 MPa in MD, the films from blends containing from 20%, 40%, 60% and 80% PPC all showed higher peak stress in MD, ranging from 30 to 45 MPa.

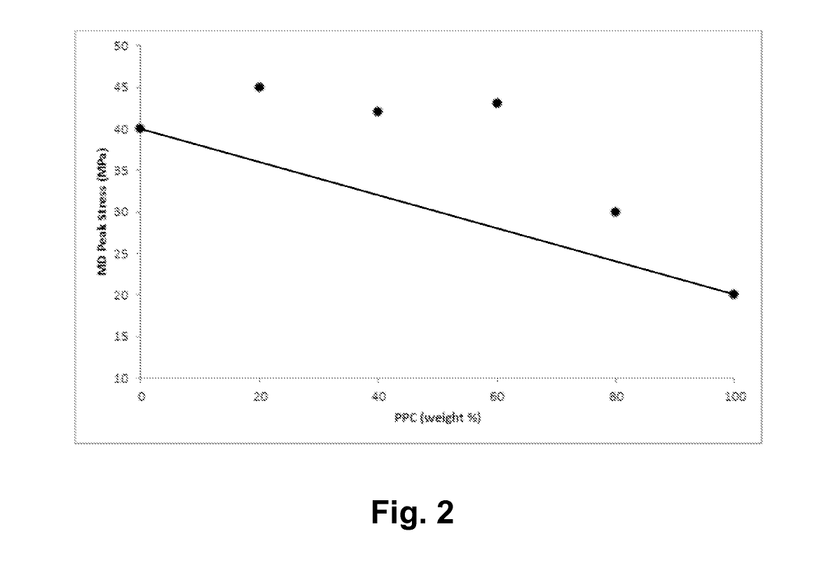

The peak stress of the films in the machine direction (MD) is shown in FIG. 2. The line connecting the peak stress of the pure LLDPE sample (at PPC wt. %=0) and the peak stress of the pure PPC sample (at PPC wt. %=100) at 40 MPa and 20 MPa, respectively, was the expected peak stress of the polymer blends containing both PPC and LLDPE. The peak stress of the compositions at 20% PPC (Example 1) was 45 MPa, at 40% PPC (Example 2) was 42 MPa, at 60% PPC (Example 3) was 43 MPa, and at 80% PPC (Example 4) was 29 MPa, all these data points are positioned well above the straight line, the line is expected from the additivity rule of polymer blends. The results showed that the peak stress in MD had surprisingly unexpected synergistic effects. These polymer blend films were also very ductile with strain-at-break values ranging from about 420 to about 560% in the MD.

TABLE 3
PE/PPC Cast Film Tensile Properties

| | | | | | | | | | | |
|---|---|---|---|---|---|---|---|---|---|---|
| | | Peak | Peak | Strain @ | | Break | Load @ | Stress @ | Strain @ | Energy per |
| | Thickness | load | stress | break | Modulus | stress | yield | yield | yield | Volume @ break |
| Code | (mil) | (gf) | (MPa) | (%) | (MPa) | (MPa) | (gf) | (MPa) | (%) | (J/cm\hat{ }3 |

MD Tensile

| | | | | | | | | | | |
|---|---|---|---|---|---|---|---|---|---|---|
| Example 1 | 1.1 | 386 | 45 | 564 | 182 | 45 | 52 | 6 | 6 | 100 |
| (2244G/Melicsea | | | | | | | | | | |
| PPC 80:20) | | | | | | | | | | |
| Example 2 | 1.1 | 357 | 42 | 453 | 234 | 42 | 62 | 7 | 5 | 76 |
| (2244G/Melicsea | | | | | | | | | | |
| PPC 60:40) | | | | | | | | | | |
| Example 3 | 1.2 | 400 | 43 | 467 | 444 | 43 | 122 | 13 | 4 | 91 |
| (2244G/Melicsea | | | | | | | | | | |
| PPC 40:60) | | | | | | | | | | |
| Example 4 | 1.1 | 255 | 30 | 421 | 1147 | 29 | 249 | 29 | 4 | 79 |
| (2244G/Melicsea | | | | | | | | | | |
| PCC) 20:80 | | | | | | | | | | |
| Comparative | 1.05 | 327 | 40 | 598 | 105 | 40 | 36 | 4 | 9 | 92 |
| Example 1 | | | | | | | | | | |
| (100% 2244G) | | | | | | | | | | |
| Comparative | 1.1 | 175 | 20 | 587 | 584 | 20 | 161 | 18 | 5 | 74 |
| Example 2 | | | | | | | | | | |
| (100% Melicsea | | | | | | | | | | |
| PPC) | | | | | | | | | | |

CD Tensile

| | | | | | | | | | | |
|---|---|---|---|---|---|---|---|---|---|---|
| 2244G/Melicsea | 1.1 | 221 | 26 | 781 | 112 | 26 | 47 | 5 | 13 | 90 |
| 80:20 | | | | | | | | | | |
| 2244G/Melicsea | 1.2 | 87 | 9 | 437 | 190 | 9 | 47 | 5 | 8 | 29 |
| 60:40 | | | | | | | | | | |
| 2244G/Melicsea | 1.1 | 125 | 14 | 478 | 487 | 14 | 95 | 11 | 3 | 48 |
| 40:60 | | | | | | | | | | |
| 2244G/Melicsea | 1.1 | 91 | 11 | 144 | 395 | 8 | 91 | 11 | 3 | 11 |
| 20:80 | | | | | | | | | | |
| 2244G (100%) | 1.05 | 280 | 34 | 825 | 87 | 34 | 37 | 5 | 9 | 119 |
| Melicsea PPC | 1.2 | 174 | 19 | 569 | 744 | 18 | 170 | 18 | 5 | 69 |
| (100%) | | | | | | | | | | |

### Example 6

### DSC (Differential Scanning Calorimeter) Method:

The various blend film samples were analyzed using a TA Instruments' Q200 Differential Scanning Calorimeter. DSC thermogram for the sample (5-10 mg) in a sealed aluminum pan was recorded in the temperature range −50° C. to 200° C. under dynamic nitrogen atmosphere using the following protocol:

Cool to 0° C.@10° C. per min, iso 2 min

Heat to +200° C.@10° C. per min, iso 2 min

Cool to −50° C.@10° C. per min, iso 2 min

Heat to +200° C. or 240° C.@10° C. per min, iso 2 min

Universal analysis NT software provided by TA Instruments was used for analyzing data.

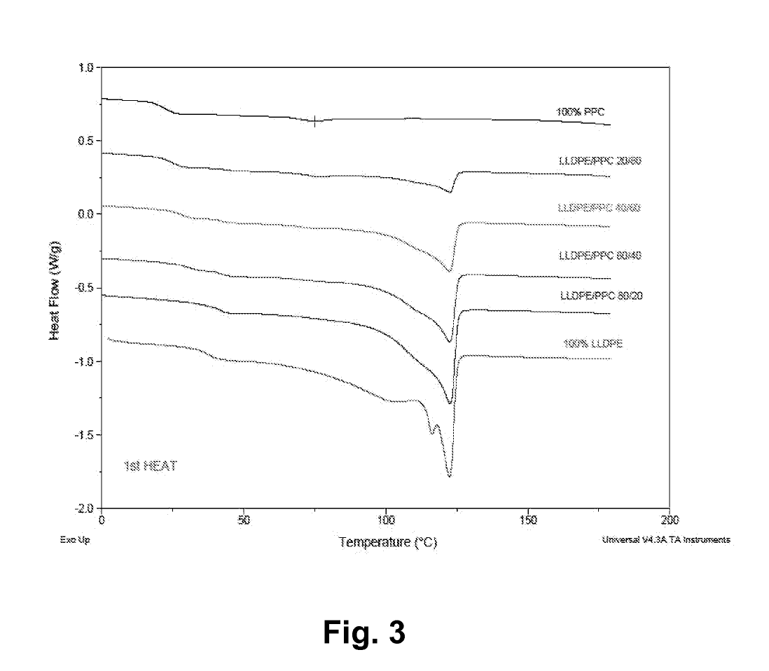

FIG. 3 shows the thermograms for the LLDPE/PPC blend films for the first heat cycle, the Tg of PPC was found to increase as the amount of LLDPE increases in the blends. The melting peak areas corresponding to polyethylene melting were found to increase as the amount of LLDPE increases as expected.

### Example 7

### Preparation of Films for SEM (Scanning Electron Microscopy)

All films were prepared identically. The direction of cut was made across the CD direction. Two pieces were cut out from different locations in the film. The samples were chilled for 1 minute in liquid nitrogen vapor to stiffen followed by rapid cutting using a chilled Teflon coated surgical razor. The sections were then mounted on aluminum SEM stubs with conductive carbon tape. Immediately the samples were placed in a plasma processing unit (Emitech Model K1050X) and lightly oxygen plasma etched for 3 minutes with O2 plasma. The plasma etch enhances the phase structure and provides improved contrast for secondary electron SEM imaging. Immediately after plasma processing was completed the samples were sputter coated with gold for 2 minutes using a Denton Desk V sputter coater. The samples were then imaged in a JEOL 6490LV SEM operating with 7 kV electron beam.

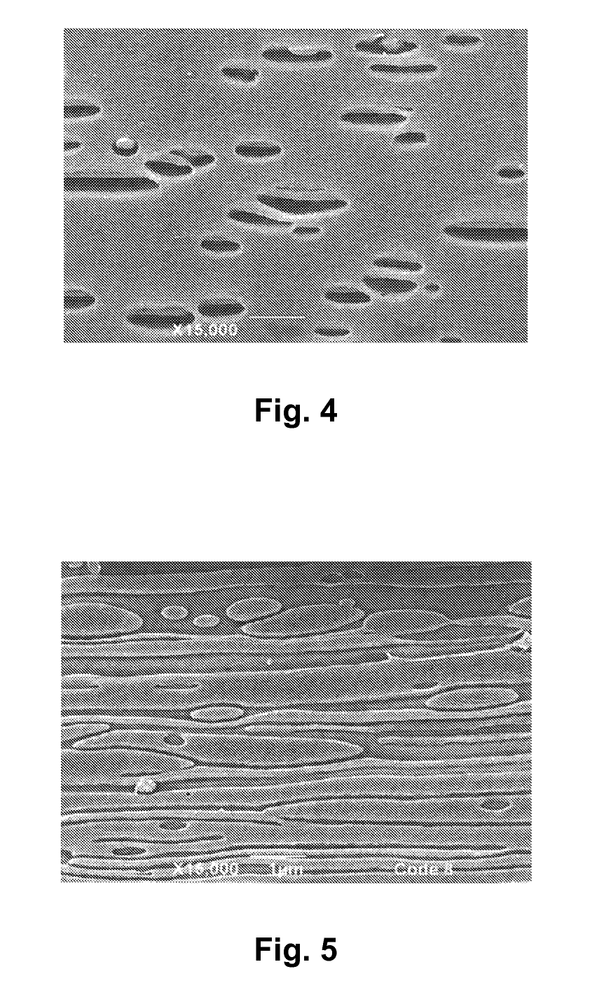

FIG. 4 shows the SEM image of a polymer blend film containing 80% (by weight) of LLDPE and 20% (by weight) of PPC (Example 1). The PPC phase is the dark, ellipsoidal-shaped dispersed phase and the LLDPE is the continuous phase. The image was taken at a magnification of 15,000×. With the carbonate groups in PPC, it is expected to be highly polar as compared to the non-polar polyethylene, therefore, the two polymers are not expected to be compatible.

FIG. 5 had a finely dispersed PPC phase, most PPC dispersions had a dimensions less than 1 μm in the longitudinal direction. Surprisingly, good dispersion was achieved at the 85:15 volume ratio of LLDPE:PPC weight ratio. With this structure, due to the complete encapsulation of PPC by LLDPE, the biodegradable PPC phase is not accessible to microorganism unless a cross section is exposed. The film is relatively stable to microorganisms.

FIG. 6 shows the cross-sectional SEM Image of a polymer blend film containing 60:40 LLDPE:PPC, as the amount of PPC was increased to 40% from 20% (FIG. 3), very interesting and surprising change in morphology was observed. The PPC phase is not in dispersed phase any more. It forms a continuous phase-like structure even as a low 40% by weight. LLDPE has a density of 0.92 g/cc, while PCC has a density of 1.26 g/cc, the volume ratio of LLDPE:PPC in this film is actually 67%:33%. This image also shows that LLDPE phase is also present as a continuous phase. Some of PPC was dispersed within the LLDPE phase, and some LLDPE phase was also dispersed in PPC phase, this shows a co-continuous phase morphology. This morphology shows unique advantage of this type of materials. Since PPC is biodegradable, even though it is only 1/3 by volume, the continuous phase would allow biodegradation by microorganisms, allowing unexpected access.

FIG. 6 exhibits the SEM micrograph of LLDPE/PPC 40:60 w/w film. The volume ratio of this film is 48%:52% LLDPE:PPC. This SEM image is also surprising that co-continuous structure was observed.

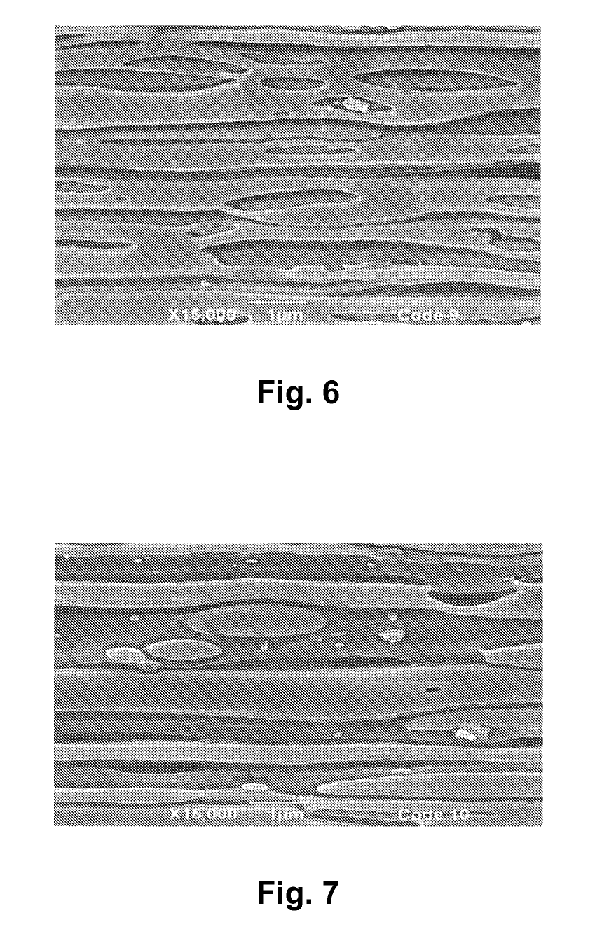

The image of LLDPE/PP 20/80 w/w is shown in FIG. 7. The volume ratio is LLDPE:PPC 25%:75%. In this case, a continuous phase of PPC is formed, while LLDPE exists as the large laminar structures or large elongated ellipsoidal structures.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A film comprising from about 10 wt. % to about 90 wt. % of at least one polyalkylene carbonate and from about 10 wt. % to about 90 wt. % of at least one polyolefin.

2. The film of claim 1, wherein the film comprises from about 10 wt. % to about 20 wt. % of at least one polyalkylene carbonate and from about 80 wt. % to about 90 wt. % of at least one polyolefin, wherein the at least one polyalkylene carbonate forms dispersed domains within a continuous phase defined by the at least one polyolefin.

3. The film of claim 1, wherein the film comprises from about 40 wt. % to about 60 wt. % of at least one polyalkylene carbonate and from about 40 wt. % to about 60 wt. % of at least one polyolefin, wherein the at least one polyalkylene carbonate and the at least one polyolefin define a co-continuous phase structure within the film.

4. The film of claim 1, wherein the polyalkylene carbonate is a polypropylene carbonate.

5. The film of claim 1, wherein the polyalkylene carbonate is a polyethylene carbonate.

6. The film of claim 1, wherein the polyalkylene carbonate is a homopolymer.

7. The film of claim 1, wherein the polyolefin is a copolymer of an α-olefin and ethylene.

8. The film of claim 1, wherein the film is free of a compatibilizer, plasticizer, or both.

9. The film of claim 1, wherein the film is free of stickiness during processing and free from block.

10. The film of claim 1, wherein the film exhibits synergistic effect on at least one of the tensile properties.

11. The film of claim 1, wherein the film has synergistic peak stress in the machine direction.

12. The film of claim 1, wherein the film has peak stress of from about 10 MPa to about 100 MPa in its machine direction.

13. The film of claim 1, wherein the film has strain-at-break of from about 400% to about 600% in its machine direction.

14. The film of claim 1, wherein the film has energy-at-break ranging from 70 to 120 J/cm³ in machine direction.

15. A packaging film comprising the film of claim 1.

16. The packaging film of claim 15, wherein the packaging film forms a wrap, a pouch, or a bag.

17. An absorbent article comprising:
a liquid permeable topsheet;
a generally liquid impermeable backsheet; and
an absorbent core positioned between the backsheet and the topsheet;
wherein the backsheet includes the film of claim 1.

18. The absorbent article as in claim 17, wherein the absorbent article defines a pad or pantiliner, wherein the film of claim 1 is a baffle film of the absorbent article.

19. The absorbent article as in claim 17, wherein the absorbent article defines a diaper or training pant, wherein the film of claim 1 is an outer cover film of the absorbent article.

20. A multi-layered film having a thickness of about 250 micrometers or less, the film comprising:
a core layer that constitutes from about 20% to about 90% of the thickness of the film, wherein the core layer comprises from about 10 wt. % to about 90 wt. % of at least one polyalkylene carbonate and from about 10 wt. % to about 90 wt. % of at least one polyolefin; and
an outer layer positioned adjacent to the core layer, wherein the outer layer contains about 50 wt. % or more of at least one polyolefin.

21. The multi-layered film of claim 20, wherein the core layer comprises from about 10 wt. % to about 20 wt. % of at least one polyalkylene carbonate and from about 80 wt. % to about 90 wt. % of at least one polyolefin, wherein the at least one polyalkylene carbonate forms pockets dispersed within a continuous phase defined by the at least one polyolefin.

22. The multi-layered film of claim 20, wherein the core layer comprises from about 40 wt. % to about 60 wt. % of at least one polyalkylene carbonate and from about 40 wt. % to about 60 wt. % of at least one polyolefin, wherein the at least one polyalkylene carbonate and the at least one polyolefin define a co-continuous phase structure within the core layer.

23. The multi-layered film of claim 20, wherein the polyalkylene carbonate is a polypropylene carbonate, a polyethylene carbonate, or a mixture thereof.

24. The multi-layered film of claim 20, wherein the polyalkylene carbonate is a homopolymer.

25. The multi-layered film of claim 20, wherein the polyolefin of the core layer, the outer layer, or both is a copolymer of an α-olefin and ethylene.

26. The multi-layered film of claim 20, wherein the core layer is free of a compatibilizer, plasticizer, or both.

27. The multi-layered film of claim 20, wherein polyolefins constitute the entire polymer content of the outer layer.

28. The multi-layered film of claim 20, wherein the outer layer further contains about 50 wt. % or less of at least one additional polymer.

29. The multi-layered film of claim 28, wherein the additional polymer is a starch polymer, aliphatic polyester, aliphatic aromatic copolyester, polylactic acid, polybutylene carbonate, polyhydroxyalkanoate, or a copolymer thereof or a mixture thereof.

30. The multi-layered film of claim 20, wherein the outer layer further contains blend of a polyolefin and a polyalkylene carbonate.

31. The multi-layered film of claim 20, wherein the core layer constitutes from about 45% to about 75% of the thickness of the film.

32. The multi-layered film of claim 20, further comprising:
a second outer layer that contains about 50 wt. % or more of at least one polyolefin.
